(19) United States
(12) Patent Application Publication
(10) Pub. No.: US 2022/0122488 A1
(43) Pub. Date: Apr. 21, 2022

(54) DISPOSABLE WRISTBAND AND THE APPARATUS FOR MANUFACTURING THE SAME

(71) Applicant: OSUNG SYSTEM CO., LTD., Gyeonggi-do (KR)

(72) Inventor: Tae-Seung SHIN, Gyeonggi-do (KR)

(21) Appl. No.: 17/138,249

(22) Filed: Dec. 30, 2020

(30) Foreign Application Priority Data
Oct. 16, 2020 (KR) 10-2020-0134209

(51) Int. Cl. G09F 3/00; G09F 3/10; G09F 3/02; B65C 9/30; B65C 9/00; B65C 5/02

(52) U.S. Cl. CPC G09F 3/005; G09F 3/10; G09F 3/02; B65C 2009/0037; B65C 9/0015; B65C 5/02; B65C 9/30

(57) ABSTRACT

A disposable wristband prevents damage to information printed on a surface even when being in contact with a drug such as alcohol. The disposable wristband may include a band body made of a fibrous material and having a predetermined length so as to be wound around a user's wrist in a band shape, a thermal label paper provided on a front surface of the band body, and an adhesive member provided on a rear surface of the band body. The thermal label paper may be processed with UV coating. The band body may be made of a nylon cloth material and may have front and rear surfaces dip-coated with polyamide. The adhesive member may be a double-sided tape and provided with a release paper. An apparatus for manufacturing the disposable wristband is also provided.

### BACKGROUND

The present disclosure relates to a disposable wristband capable of preventing damage to information printed on a surface even when being in contact with a drug such as alcohol, and to an apparatus for manufacturing the same.

In general, medical facilities such as hospitals and clinics are using a patient wristband that displays patient management information including patient's name, age, sex, blood type, and treatment department.

Further, various kinds of wristbands are provided to visitors in performance halls, buffet restaurants, conference halls, and particular places to simply offer a variety of information such a seating position and admission permission.

Conventional disposable wristbands have been disclosed in Korean Utility Model Publication No. 1998-016229 entitled ‘wristband with a note-insertion part’ and Korean Patent Publication No. 2013-0083448 entitled ‘wristband, continuous body of wristbands, and method for winding wristband’.

For example, as shown in (a) of FIG. 1, a disposable wristband includes a base film 11, an adhesive layer 12, and a display film 13. Cutting the base film 11 along a cutting line C, the adhesive layer 12 is exposed, and the wristband can be worn on the wrist.

In addition, as shown in (b) of FIG. 1, the display film 13 has a handwriting portion 13a and an advertisement portion 13b. An administrator can directly write wearer's unique information in the handwriting portion 13a, or attach or insert a sticker printed with such information to or into the handwriting portion 13a.

However, most of the conventional disposable wristbands generally use a coated paper as the base film 11 or the display film 13. Therefore, there is a problem with cuts or injuries to a wrist on a sharp side of the paper.

In case of alternatively using vinyl or synthetic resin, which is not easily torn and resistant to moisture, skin problems may occur. Especially, serious problems may occur for people with sensitive skin or children when worn for a long time.

In a certain case, artificial leather is used instead of the base film 11 or the display film 13, and a dot button is used as a detachable means. However, in this case, there is a problem in that the manufacturing cost is increased and the wrist is pressed by the dot button, so that the fit is not good.

Further, such conventional materials make it difficult to print the wearer's information on the display film 13 through a printer. Even if printing is performed, the printed information may be easily spread or erased. This causes an inconvenience of having to directly write the information.

In order to solve the above-described conventional problems, Korean Patent No. 10-1477633 discloses a ‘disposable wristband’ as shown in FIG. 2.

However, when this disposable wristband 20 is used in a hospital, etc., there is a problem that the contents printed on a unique information portion 21a and an additional information portion 21b of the wristband are frequently erased due to contact with drugs such as alcohol.

### SUMMARY OF THE INVENTION

The present disclosure provides a disposable wristband capable of preventing damage to information printed on a surface even when being in contact with a drug such as alcohol, and to an apparatus for manufacturing the same.

According to embodiments of the present disclosure, a disposable wristband may include a band body made of a fibrous material and having a predetermined length so as to be wound around a user's wrist in a band shape, a thermal label paper provided on a front surface of the band body, and an adhesive member provided on a rear surface of the band body. The thermal label paper may be processed with UV coating. The band body may be made of a nylon cloth material and may have front and rear surfaces dip-coated with polyamide. The adhesive member may be a double-sided tape and provided with a release paper.

In the disposable wristband, a first support body or a second support body made of an air-permeable material may be formed on the rear surface of the band body. In addition, the release paper may be detachably provided on the front surface of the thermal label paper that is printed by a thermal printer.

According to embodiments of the present disclosure, an apparatus for manufacturing a disposable wristband may include a base provided with at least one driving motor operated by a control signal from a controller; an unwinding roller rotatably installed on the base and having a band body wound in a roll form thereon and transferred in one direction when unwound by rotation, wherein the band body is made of a nylon cloth material, has front and rear surfaces dip-coated with polyamide, and includes an adhesive member provided on the rear surface; a label supplier rotatably installed on the base and supplying a UV-coated thermal label paper to the front surface of the band body being transferred; a release paper provider rotatably installed on the base and providing a release paper to a front surface of the thermal label paper; and a winding roller rotatably installed on the base and winding in a roll form the band body transferred from the unwinding roller, wherein the band body is provided with the thermal label paper and the release paper.

In the apparatus, the label supplier may include a label roller supplying the thermal label paper to the band body, and a coating member processing UV coating on the front surface of the thermal label paper to be provided on the band body.

### BRIEF DESCRIPTION OF THE DRAWINGS

FIGS. 1 and 2 are views showing conventional wristbands.

FIG. 3 is a view showing a disposable wristband according to an embodiment of the disclosure.

FIG. 4 is a cross-sectional view taken along the line A-A′ of FIG. 3.

FIG. 5 is a view showing a band body of a disposable wristband according to another embodiment of the disclosure.

FIG. 6 is a schematic view showing an apparatus for manufacturing a disposable wristband according to an embodiment of the disclosure.

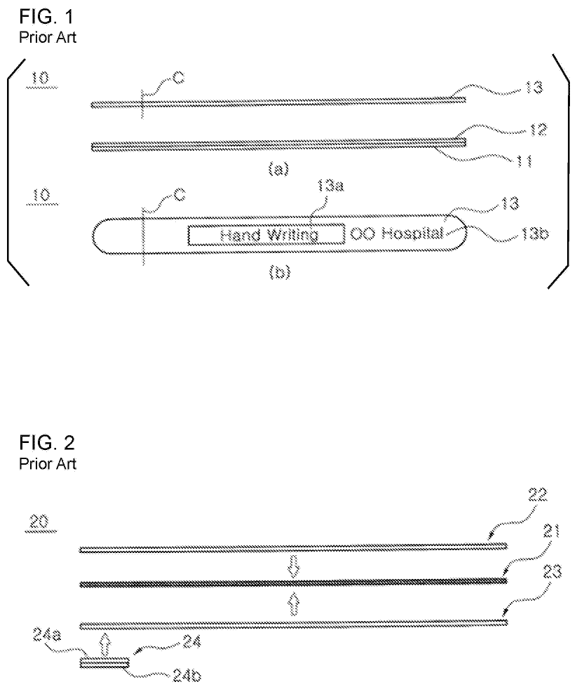

### DETAILED DESCRIPTION OF THE INVENTION

Hereinafter, embodiments of the present disclosure will be described in detail with reference to the accompanying drawings. The present disclosure may, however, be embodied in many different forms and should not be construed as being limited to the embodiments set forth herein. Rather, these embodiments are provided so that the disclosure will be thorough and complete and will fully convey the scope of the disclosure to those skilled in the art.

In the following description of embodiments, techniques that are well known in the art and not directly related to the present disclosure are not described. This is to clearly convey the subject matter of the present disclosure by omitting an unnecessary explanation. For the same reason, some elements in the drawings are exaggerated, omitted, or schematically illustrated. Also, the size of each element does not entirely reflect the actual size. In the disclosure, the same or corresponding elements are denoted by the same reference numerals.

Now, a disposable wristband (hereinafter, simply referred to as ‘wristband’) according to an embodiment of the disclosure will be described in detail with reference to the accompanying drawings.

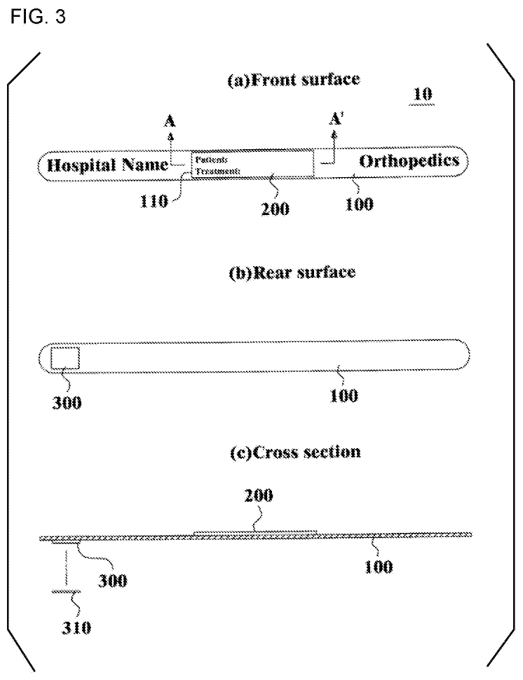

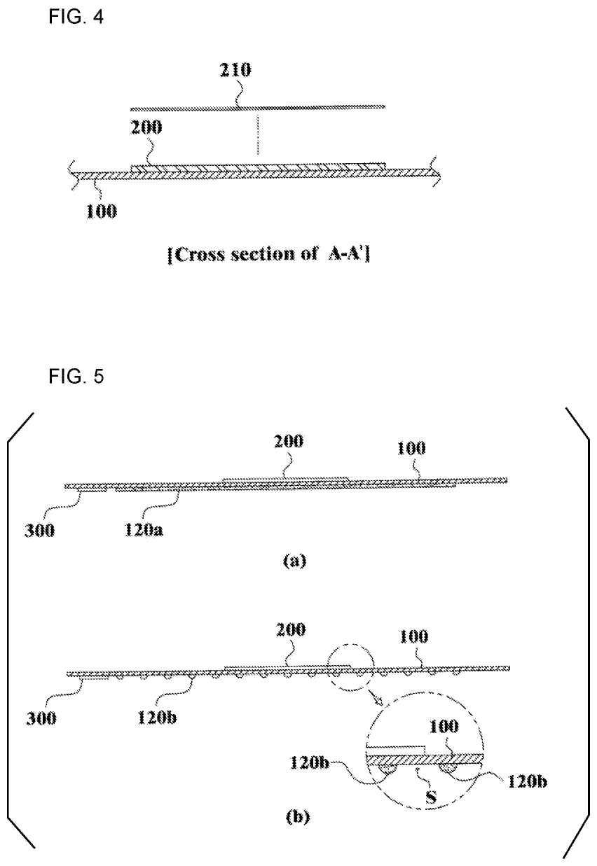

As shown in FIGS. 3 to 4, a wristband 10 includes a band body 100, a thermal label paper 200, and an adhesive member 300.

The band body 100 has a predetermined length as shown. In use, both ends of the band body 100 are attached to each other in the form of a band to be worn on a user's wrist.

For example, the band body 100 is made of a fibrous material. The fibrous material is available for printing (not thermal printing), has a good fit, does not have a fear of cutting a wrist by a sharp side, is harmless to a human body, does not cause skin troubles after wearing, and has excellent durability and moisture resistance.

In order to satisfy these conditions, the band body 100 uses a nylon cloth, which refers to ‘cloth’ made of nylon fibers. Herein, ‘cloth’ means a fabric.

Nylon is a generic term for synthetic polymer polyamide, which has links formed of an amide bond —CONH— and is a chain-shaped polymer.

Unlike normal cloth, nylon cloth has excellent printability and thus allows usage information to be printed on a front or rear surface of the nylon cloth by using a printer capable of printing ink. The usage information is, for example, specific information such as hospital information.

In the disclosure, it is preferable that the usage information is not directly printed by a user or an administrator, but printed on the surface of the band body 100 in advance by a manufacturing apparatus 20 to be described later.

That is, as shown, a hospital name or a name of a treatment department (e.g., internal medicine, or obstetrics and gynecology, etc.) is printed on the surface of the band body 100 except for a blank portion 110 to which the thermal label paper 200 is attached. This conveniently informs the hospital and department in which a user (or patient) wearing the wristband 10 is being treated.

Meanwhile, as shown in FIG. 5, on a lower surface of the band body 100, a first support body 120a or a second support body 120b made of a breathable material such as a sponge may be formed. The first support body 120a or the second support body 120b prevents the band body 100 from damaging the skin of the user wearing the wristband 10.

As shown, the first support body 120a is formed under the band body 100 to have a predetermined length in a longitudinal direction. The first support body 120a is formed of an air-permeable material having fine pores, absorbs sweat generated from the skin of the wearer, and prevents skin damage due to direct contact between the band body 100 and the skin.

In addition, the second support body 120b is formed of the same material as the first support body 120a. However, contrary to the first support body 120a, the second support body 120b is composed of a plurality of embossing protrusions which are arranged at regular intervals under the band body 100 while forming space portions S. The second support body 120b has air permeability itself and additionally provides air permeability through the space portion S. Therefore, the second support body 120b can minimize damage to the skin when a user having very sensitive skin due to a burn or the like wears the wristband 10.

As such, the wristband 10 of the disclosure is worn by a user (i.e., a patient) using a hospital. Among users, there may be users who need skin protection due to burns or skin diseases, and in this case, it is important that the wristband 10 has air permeability. The first support body 120a or the second support body 120b formed under the band body 100 not only improves air permeability, but also prevents skin damage when the wristband 10 is worn.

The thermal label paper (or thermal printing label paper) 200 allows an administrator (i.e., hospital personnel) to print user information on a user wearing the wristband 10 through a thermal printer (not shown).

The thermal label paper 200 attached to the blank portion 110 is a recording paper coated with a colorless dye and an organic acid. When the thermal label paper 200 is heated through a thermal printer, the colorless dye and the organic acid are melted and mixed with each other to develop color. A base paper of the thermal label paper 200 is formed of oil paper, which may be a recyclable polypropylene paper.

The colorless dye is formed by atomizing electron-donating colorless leuco dye (such as crystal violet lactone) and electron-accepting colorant (phenolic acid substances) and applying them to the base paper together with a binder.

A detachable release paper 210 may be provided on the surface of the thermal label paper 200. When printing user information on the thermal label paper 200, the administrator detaches the release paper 210 from the thermal label paper 200, inserts the thermal label paper 200 into the thermal printer, and inputs user information through an input unit of the thermal printer.

The release paper 210 may be reattached to the thermal label paper 200 on which the user information is printed. Through this, it is possible to prevent the printed user information from being erased by hospital drugs such as alcohol. However, it is desirable to use the thermal label 200 having a UV-coated surface so as to prevent the printed user information from being damaged.

The adhesive member 300 is provided on the rear surface of the band body 100. When the band body 100 is worn on the user's wrist in a band shape while both ends of the band body 100 are overlapped with each other, the adhesive member 300 allows both ends of the band body 100 to be adhered to each other.

The adhesive member 300 may be provided at one end or both ends of the band body 100. The outer surface of the adhesive member 300 is not exposed to the air by the release paper 310 attached thereto. In use, the release paper 310 is detached from the adhesive member 300, and the adhesive member 300 is adhered to each other at both ends of the band body 100 or adhered to the surface of the band body 100 at one end of the band body 100.

Hereinafter, an apparatus for manufacturing a disposable wristband (hereinafter, simply referred to as a “manufacturing apparatus”) according to an embodiment of the disclosure will be described in detail with reference to the accompanying drawings.

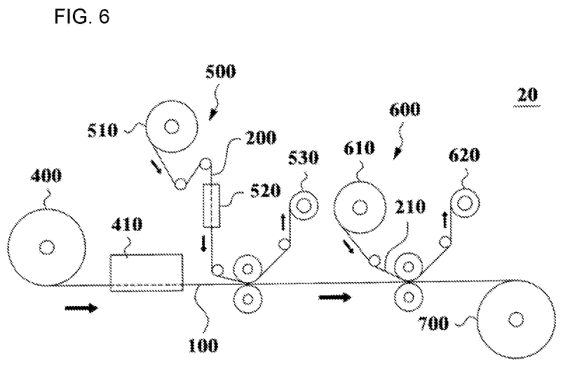

As shown in FIG. 6, the manufacturing apparatus 20 includes a base (not shown), an unwinding roller 400, a label supplier 500, a release paper provider 600, a winding roller 700, and a controller (not shown). The base (not shown) is a structure composed of a plurality of horizontal frames and vertical frames which are fastened to each other by bolts, screws, and the like, and offers a basis where the unwinding roller 400, the label supplier 500, the release paper provider 600, and the winding roller 700 are installed. The controller (not shown) is installed on the base or provided spaced apart from the base, and is configured to control the unwinding roller 400, the label supplier 500, the release paper provider 600, and the winding roller 700 by transmitting a control signal to each of them via a wired cable or wireless communication.

The unwinding roller 400 has a driving motor (not shown) and is rotatably installed on the base (not shown). The band body 100 is wound in the form of roll around the unwinding roller 400. By the rotation of the winding roller 700, the band body 100 wound around the unwinding roller 400 is unwound and transferred in one direction (i.e., a direction toward the winding roller 700).

The unwinding roller 400 includes a printing member 410 installed on the base. In response to a control signal received from the controller (not shown), the printing member 410 may print hospital information or the like on the surface of the band body 100 being transferred (See FIG. 3).

The printing member 410 may perform printing on one or both end portions of the surface of the band body 100 in response to a control signal from the controller so that the blank portion 110 is formed in the central portion of the surface of the band body 100.

The label supplier 500 includes a label roller 510, a coating member 520, and a first recovery roller 530. The label supplier 500 attaches the UV-coated thermal label paper 200 to the blank portion 110 formed on the surface of the band body 100 being transferred.

Each of the label roller 510 and the first recovery roller 530 has a driving motor (not shown) and is rotatably installed on the base (not shown). The thermal label paper 200 wound in a roll form on the label roller 510 is unwound from the label roller 510 by the pull of the first recovery roller 530 and attached to the surface of the band body 100 (See FIG. 3).

In order to attach the thermal label paper 200 to the band body 100, a pair of pressing rollers may be preferably used as shown.

The coating member 520 is fixedly installed on the base (not shown). The thermal label paper 200 unwound from the label roller 510 by the first recovery roller 530 enters the coating member 520. Then, the coating member 520 processes UV coating on the surface of the thermal label paper 200.

The release paper provider 600 includes a supply roller 610 and a second recovery roller 620. Using a pair of pressing rollers, the release paper provider 600 attaches the release paper 210 to the surface of the thermal label paper 200 adhered to the blank portion 110 of the band body 100.

The release paper 210 wound in the form of a roll on the supply roller 610 is unwound from the supply roller 610 by the rotation of the second recovery roller 620. Then, the release paper 210 is detachably attached to the surface of the thermal label paper 200 by the pressing rollers (See FIG. 4).

The second recovery roller 620 is installed on the base (not shown) so as to be rotatable by a driving motor (not shown). The second recovery roller 620 provides rotational force so that the release paper 210 can be unwound while the supply roller 610 rotates, and also recovering the original paper from which the release paper 210 is separated.

The second recovery roller 620 is installed on the base so as to be rotatable by a driving motor. The second recovery roller 620 provides rotational force for the release paper 210 to be unwound from the supply roller 610, and recovers a base paper from which the release paper 210 is separated.

The winding roller 700 is installed on the base at a position opposite to the unwinding roller 400. The winding roller 700 rotates by a drive motor to provide a rotational force so that the band body 100 wound around the unwinding roller 400 can be unwound. In addition, the winding roller 700 winds the band body 100 provided with the thermal label paper 200 and the release paper 210 in a roll form.

Meanwhile, although not shown, the band body 100 provided with the thermal label paper 200 and the release paper 210, which is wound in a roll form on the winding roller 700, is cut to a predetermined length by a cutting machine (not shown) and then wrapped to be shipped.

As described above, the wrist band 10 according to the present disclosure has the UV-coated thermal label paper 200 provided on the surface of the band body 100 worn on the user's wrist. When user information is printed on the thermal label paper 200, it is possible to prevent a defect in that the printed user information is erased by hospital drugs such as alcohol.

While the present disclosure has been particularly shown and described with reference to an exemplary embodiment thereof, it will be understood by those skilled in the art that various changes in form and details may be made therein without departing from the scope of the present disclosure as defined by the appended claims.

What is claimed is:

1. A disposable wristband comprising:
a band body made of a fibrous material and having a predetermined length so as to be wound around a user's wrist in a band shape;
a thermal label paper provided on a front surface of the band body; and
an adhesive member provided on a rear surface of the band body,
wherein the thermal label paper is processed with UV coating, the band body is made of a nylon cloth material and has front and rear surfaces dip-coated with polyamide, and the adhesive member is a double-sided tape and provided with a release paper.

2. The disposable wristband of claim 1, wherein a first support body or a second support body made of an air-permeable material is formed on the rear surface of the band body, and
wherein the release paper is detachably provided on the front surface of the thermal label paper that is printed by a thermal printer.

3. An apparatus for manufacturing a disposable wristband, comprising:
a base provided with at least one driving motor operated by a control signal from a controller;
an unwinding roller rotatably installed on the base and having a band body wound in a roll form thereon and transferred in one direction when unwound by rotation, wherein the band body is made of a nylon cloth material, has front and rear surfaces dip-coated with polyamide, and includes an adhesive member provided on the rear surface;
a label supplier rotatably installed on the base and supplying a UV-coated thermal label paper to the front surface of the band body being transferred;
a release paper provider rotatably installed on the base and providing a release paper to a front surface of the thermal label paper; and
a winding roller rotatably installed on the base and winding in a roll form the band body transferred from the unwinding roller, wherein the band body is provided with the thermal label paper and the release paper.

4. The apparatus of claim 3, wherein the label supplier includes:
a label roller supplying the thermal label paper to the band body; and
a coating member processing UV coating on the front surface of the thermal label paper to be provided on the band body.
